(19) United States
(12) Patent Application Publication
(10) Pub. No.: US 2006/0173525 A1
(43) Pub. Date: Aug. 3, 2006

(54) Methods and systems for deploying luminal prostheses

(75) Inventors: Robert S. Behl, Palo Alto, CA (US); Alexander L. Huang, Menlo Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Percutaneous Systems, Inc., Mountain View, CA (US)

(21) Appl. No.: 11/346,606

(22) Filed: Feb. 1, 2006

Related U.S. Application Data
(60) Provisional application No. 60/649,848, filed on Feb. 2, 2005.

(51) Int. Cl. A61F 2/06

(52) U.S. Cl. 623/1.11

(57) ABSTRACT

Stents and other luminal prostheses may be introduced to body lumens, such as ureters, within a lubricious sleeve. The sleeve may be packed within a distal end of the stent so that it may be everted and withdrawn to cover an exterior surface of the stent as the stent is advanced. The stent is typically advanced using a separate pusher member which is engaged against a proximal end of the stent. The stent may be anchored within the body lumen by deforming or otherwise deploying anchor structures at either or both ends of the stent.

### CROSS-REFERENCES TO RELATED APPLICATIONS

The present application is a non-provisional of U.S. patent application Ser. No. 60/649,848 (Attorney Docket No. 021807-001400US), filed Feb. 2, 2005, the full disclosure of which is incorporated herein by reference.

### BACKGROUND OF THE INVENTION

1. Field of the Invention

The present invention relates generally to medical apparatus and methods for using such apparatus. In particular, the present invention relates to systems and methods for delivering stents and other luminal prostheses to target locations in body lumens.

Urinary stents are used to provide urinary tract patency in a variety of circumstances, including bypassing obstructions and strictures and preserving patency after operations. Most commonly, stents are placed to bypass kidney stones in the ureter, the duct which drains urine from the kidney to the urinary bladder. The stents, referred to as ureteral stents, are thermoplastic or silicone tubes with shaped ends which anchor the stent in the kidney and bladder, respectively.

The placement of ureteral stents is generally safe, but in some instances can cause damage to the endothelium of the ureter, and in the worst cases can perforate the ureteral wall. The placement procedures generally require fluoroscopic or other advanced imaging techniques and that the patient be under a general anesthesia. For those reasons, the procedures must be performed in a hospital or well-equipped clinical location.

Conventional ureteral stent placement typically is done through a cystoscope which is advanced into the bladder through the urethra. The cystoscope is used to locate the uretero-vesical orifice, and a guidewire is advanced from the cystoscope into the ureter. The procedures typically begin with a very flexible guidewire, typically having a diameter of about 1 mm, which presents little risk of damage to the ureter. Often, however, the initial guidewires are not sufficiently stiff to pass obstructions, such as impacted kidney stones. To increase column strength and pushability, a small sleeve or guiding catheter can be placed over the initial wire to provide sufficient support for the guidewire to advance past the obstruction. The risk of damage to the ureter, however, is increased.

Once the guidewire has reached the kidney, it may be necessary to exchange the initial wire for a stiffer wire. The ureteral stent will then be introduced over the selected wire. Typical ureteral stents have a tapered leading end with a lumen. The leading end is typically coiled and the trailing end is square-cut. Both ends will be straightened as they are introduced over the wire, and a pusher tube will be introduced coaxially over the wire in order to push and advance the stent through the ureter and into the kidney. Once the stent is properly placed with its leading end in the kidney and its trailing end in the bladder, the guidewire is removed allowing each end to return to a pre-shaped anchor configuration.

In the case of severe obstructions in the ureter, it may be necessary to exert a relatively large force on the stent to bypass the obstruction. While, with a stiff guidewire in place it is possible to advance the stent pass nearly any type of obstruction, the need to exert larger forces significantly increases the potential for injuring the ureter, particularly when passing impacted stones.

For these reasons, it would be desirable to provide improved systems and methods for introducing stents and other luminal prostheses into ureters and other body lumens. In particular, it would be desirable to provide methods and systems for advancing such prostheses through the body lumens and past obstructions, strictures, and other problematic regions while minimizing the risk of damage to the body lumen and luminal wall. For example, it would be desirable to be able to place a ureteral stent with minimum risk of trauma or damage to the ureteral wall. It would be further desirable to be able to perform ureteral stent placement without the need for general anesthesia and/or advanced fluoroscopic imaging. Even more preferably, it would be desirable to provide such methods and systems which would allow stent and other prosthesis placement in locations other than an operating room, and in some cases even outside the hospital. At least some of these objectives will be met by the inventions described hereinbelow.

2. Description of the Background Art

Ureteral stents and stent deployment systems are described in U.S. Pat. Nos. 4,531,933; 4,738,667; 4,790,810; and 4,913,683, and in Hepperlen et al. (1978) J. Urol. 119:731-734; Doppman et al. (1979) Radiology 132:735-737; and Doherty (1984) JAMA 252:1108-1109. The use of an everting sleeve composed of thin, tensilized polytetrafluoroethylene for introducing catheters to body lumens is described in U.S. Pat. Nos. 5,531,717; 5,676,688; 5,711,841; 5,897,535; 6,007,488; 6,240,968; and EP605427B1. Other catheters employing everting sleeves for a variety of purposes are described in commonly assigned, copending application nos. 10/794,337 (Attorney Docket No. 021807-000300US), filed on Mar. 5, 2004, 10/794,317 (Attorney Docket No. 021807-000400US), filed on Mar. 5, 2004, 10/886,886 (Attorney Docket No. 021807-000800US), filed on Jul. 7, 2004, and 10/993,631 (Attorney Docket No. 021807-001300US), filed on Nov. 19, 2004, the full disclosure of which are incorporated herein by reference.

### BRIEF SUMMARY OF THE INVENTION

The present invention provides methods and systems for delivering prostheses to body lumens. The prostheses may be any one of a wide variety of luminal devices, such as stents, grafts, catheters, cannulas, drain tubes, and other tubular structures, most commonly being structures intended to create and/or maintain patency of the body lumen. The body lumens may be any natural or created passage through the body or body tissue. While the following description is directed specifically at delivering stents to the ureter, the invention will also apply to delivering the other prostheses mentioned above to other body lumens, such as the urethra, other regions of the urinary tract, the biliary duct, the hepatic duct, the cervix, the fallopian tubes, the gastrointestinal tract, blood vessels, and the like. The body lumens may also be created body lumens, such as fistulas, access tracts, and the like.

The prostheses of the present invention are introduced using a lubricious sleeve which remains stationary as the prosthesis is advanced to protect the luminal wall. As the prosthesis is advanced, the lubricious sleeve will usually evert over a distal tip or end of the prosthesis so that the sleeve lays down over or “tracks” the luminal wall to provide the desired protective layer to inhibit injury to the luminal wall. Usually, the sleeve is stored in a lumen of the prosthesis, and the sleeve is pulled or everted out of a distal tip of the prosthesis as the prosthesis is advanced. Such tracking of the sleeve also helps the prosthesis move past stones, strictures, and any other obstructions which may be present in the body lumen.

The prosthesis is usually advanced by a push member which engages a rear or proximal end of the prosthesis. Once in place, the pusher can be withdrawn proximally, leaving the prosthesis in place at the desired location within the body lumen. An anchor structure disposed and at least one end of the prosthesis will usually be deployed in order to immobilize the prosthesis within the body lumen or within a body cavity open to the lumen. The anchor structure can be any one of a variety of mechanisms. In a particular embodiment, the anchor structure comprises a pre-shaped portion or end of the prosthesis so that the prosthesis may be constrained into a generally straight or linear configuration for introduction into and through the body lumen. Once in place, the constraint may be removed to allow the pre-shaped portion to return to its anchoring or deployed configuration. Alternatively, the sleeve may be generally straight or linear in its unconstrained configuration, and a deflecting wire or other mechanism introduced into a lumen of the prosthesis to deflect it and form the desired anchor structure. Optionally, the lubricious sleeve may be removed from over the prosthesis after the prosthesis has been positioned, either before or after the anchor structure(s) have been deployed.

The lubricious sleeve is preferably evertable and will typically comprise a polymeric tube, more particularly being a thin walled polymeric tube made from a lubricious polymer or a polymer which may be lubricated on at least one side. The polymeric tube typically has a length in the range from 5 cm to 90 cm, preferably from 10 cm to 35 cm, an inner diameter (for a single lumen) in the range from 2 mm to 12 mm, preferably in the range from 2 mm to 6 mm, and a wall thickness in the range from 0.01 mm to 0.05 mm, preferably from 0.02 mm to 0.04 mm. Exemplary polymers for the polymeric tube include polytetrafluoroethylene (PTFE), polyethylene (PE), perfluoroalkoxy (PFA), polyurethane (PU), perfluoromethylvinylether (MFA), perfluoroprophylvinylether (PPVE), and copolymers thereof. Preferred polymers include tensilized PTFE/PPVE copolymers.

Thus, according to a first method of the present invention, a self-anchoring tubular stent or other prosthesis is deployed in a body lumen. The stent is advanced in a distal direction through a stationary lubricious sleeve. At least one anchoring structure is formed at a distal end of the stent after the stent has reached the target location in the body lumen. Advancing the stent typically comprises pushing a pusher member against a proximal end of the stent, and the pusher member is typically separated from the stent and withdrawn in a proximal direction after the stent has reached the target location. Preferably, advancing the stent comprises everting the lubricious sleeve over a distal end of the stent as the stent is advanced. In this way, the lubricious sleeve is deployed between the exterior of the stent and the inner wall of the body lumen as the stent is advanced. Usually, a distal end of the lubricious sleeve (i.e., the end which first emerges from a lumen of the stent and everts over the distal end of the stent) is held stationary relative to the body location as the stent is advanced. The distal end of the lubricious sleeve may be held by an outer positioning tube which remains stationary within the body lumen. When such an outer positioning tube is used, the stent (as well as the pusher member) is usually advanced through a central passage in the outer positioning tube. Optionally, the lubricious sleeve may be removed from over the stent either by pulling on a proximal end or a distal end of the lubricious sleeve. When pulling on a proximal end, the sleeve will be withdrawn through the lumen of the stent with the distal or leading end of the sleeve being pulled proximally past an exterior surface of the stent before it is then drawn back into the lumen of the stent. When pulling on the distal end of the lubricious sleeve (e.g., using the outer positioning tube), the proximal or trailing end of the sleeve will be drawn distally through the stent lumen and proximally back over the exterior of the stent. In either case, the lubricious nature of the sleeve allows its withdrawal without significantly disturbing the position of the stent or other prosthesis within the body lumen.

In a second method according to the present invention, a ureteral stent is introduced to a ureter by positioning the stent in an outer positioning tube disposed at a ureteral orifice in a patient's bladder. The stent is pushed on its proximal end to advance the stent into the ureter, and a lubricious sleeve everts from a lumen of the stent over the distal end of the stent so that the sleeve is laid stationary over the wall of the ureter to provide a protective layer between the stent and the ureteral wall. Pushing on the stent is stopped after a distal portion of the stent has entered a kidney. At least one anchoring structure on the distal portion of the stent is deployed or formed to anchor the distal portion within the kidney.

In preferred aspects of this second method, the sleeve is everted while the distal end of the sleeve is immobilized relative to the ureter. While the sleeve is immobilized, the stent is advanced or pushed against an everting fold of the sleeve in order to distally advance the fold while minimizing the trauma to the ureteral wall. The distal end of the sleeve is typically immobilized by attachment to the outer positioning tube, where the outer positioning tube is held stationary relative to the ureteral wall. After the stent is properly positioned, the distal end of the sleeve may be released from the outer positioning tube, allowing the proximal end of the sleeve to be pulled and withdrawn from the stent. Alternatively, the attachment between the outer positioning tube and the stent may be maintained so that the sleeve is withdrawn as the outer positioning tube is withdrawn from the ureter.

The anchoring structure may be formed by either of the techniques described above. Again, the anchoring structure may be pre-shaped and released from constraint so that it assumes a non-linear configuration which acts as an anchor within the kidney (and optionally at the other end of the stent within the bladder). Alternatively, the stent may have a generally linear configuration which may be deformed into an anchor using a pre-shaped wire or other shaping tool.

The present invention still further provides stent deployment systems comprising a tubular stent and an evertable, lubricious sleeve. The tubular stent has a proximal end, a distal end, a lumen therethrough, an exterior surface, and a deployable anchor structure at at least the distal end. An evertable, lubricious sleeve is adapted to emerge from the distal end of the stent and evert over the exterior surface as the stent is advanced through a ureter or other body lumen. For ureteral deployment, the stent will typically not be radially expandable. In other applications, however, the tubular stent (or at least a portion thereof) may be expandable, including both self-expanding structures and balloon-expandable structures. Preferably, the anchor structure comprises a pre-shaped distal end of the stent having a non-linear configuration when unconstrained and a generally linear configuration when constrained. In the exemplary embodiments, the stent will further comprise a proximal anchor structure including a similar or dissimilar pre-shaped proximal end. In alternative stent structures, the anchor structure may have a generally linear configuration which is deformed using a separate shaping wire or other shaping tool.

The systems of the present invention will usually further comprise a pusher member, where the pusher member preferably has a central passage. The systems may still further comprise a restraining wire receivable through the central passage of the pusher member as well as the lumen of the stent. The wire will be adapted to maintain the pre-shaped regions of the stent in a generally linear configuration. Thus, the restraining wire could be straight, could have a curve which is equal and opposite to the curve of the pre-shaped region of the stent, or the like. Usually, the restraining wire will be a core wire with a length generally equal to the combined lengths of the pusher member and the stent. Alternatively, the restraining member could be an outer tubular member which is received over both the pusher member and the stent to hold the pre-shaped end(s) of the stent in a generally linear configuration.

The stent deployment system of the present invention may further comprise an outer positioning tube having a central passage which receives the tubular stent and optionally the tubular pusher member. The evertable lubricious sleeve may be releasably attached to a distal end of the outer positioning tube so that the stent can be advanced distally through the outer positioning tube while the sleeve is immobilized and everted from the lumen of the stent to track over the exterior of the stent.

In all methods and systems described above, the stent and deployment system may be introduced without use of a separate guidewire. It is possible, however, that the stent and/or pusher member could also be introduced over a conventional guidewire is that is desired. In such cases, the conventional guidewire could act as the constraining wire for maintaining the pre-shaped end(s) of the stent in a generally linear configuration during delivery through the body lumen.

### BRIEF DESCRIPTION OF THE DRAWINGS

FIG. 1 is an exploded view of a stent deployment system constructed in accordance with the principles of the present invention.

FIG. 2 is an assembled view of the stent deployment system of FIG. 1 shown in partial section with portions broken away.

FIGS. 3A and 3B are schematic illustrations of deployment of the evertable, lubricious sleeve from the distal end of the stent deployment system.

FIGS. 4A-4D are more detailed views of the deployment of the everting lubricious sleeve of the present invention taken generally along line 4-4 in FIG. 2.

FIG. 5 illustrates a first anchor structure deployment mechanism in accordance with the principles of the present invention.

FIG. 6 illustrates a second anchor deployment mechanism constructed in accordance with the principles of the present invention.

FIG. 7 illustrates a third anchor deployment mechanism constructed in accordance with the principles of the present invention.

FIG. 8 illustrates a fourth anchor deployment mechanism constructed in accordance with the principles of the present invention.

FIG. 9 illustrates use of the stent deployment system of the previous figures for the introduction of a stent using a cystoscope to access a ureter.

FIGS. 10A and 10B show advancement of the distal end of the deployment system past a kidney stone in a ureter.

FIGS. 11A-11C illustrate deployment of an anchor structure at the distal end of the stent within the kidney.

### DETAILED DESCRIPTION OF THE INVENTION

Systems and methods of the present invention are useful for deploying stents and other prostheses into any natural or created body lumen of a patient where it is desired to temporarily or permanently reinforce the lumen, provide a drainage structure, occlude the lumen, or for any other purpose. Most commonly, the systems and methods of the present invention will be used to place a ureteral stent in a ureter to maintain a drainage path between the kidney and the bladder, as described in detail in the following description.

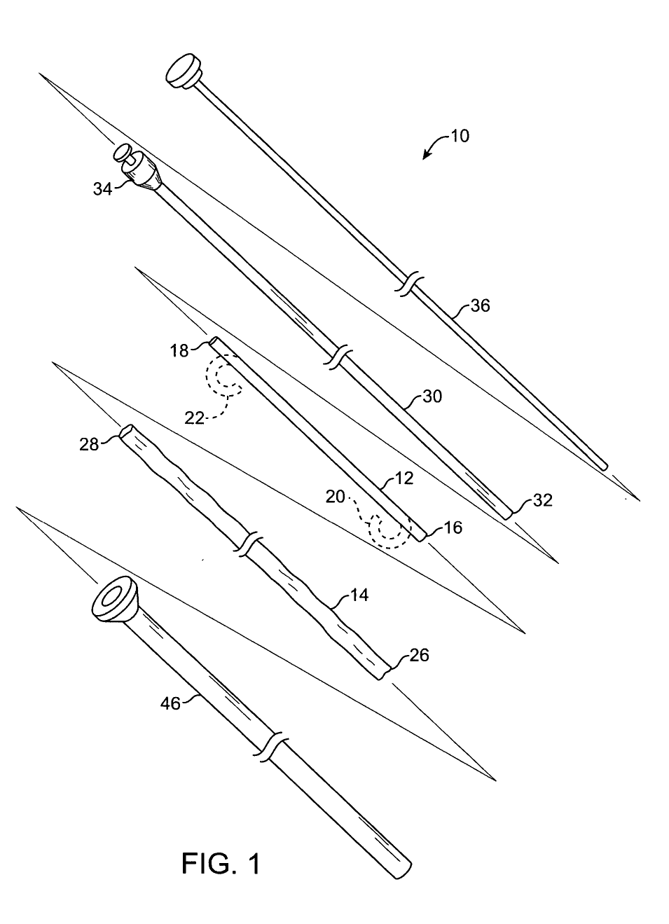

A stent deployment system 10 constructed in accordance with the principles of the present invention includes a stent 12 and an evertable, lubricious sleeve 14, as illustrated in FIG. 1. The stent will be tubular and have a central lumen which passes from a distal end 16 to a proximal end 18 of the stent. At least the distal end 16, and preferably the proximal end 18 as well, will define or be attached to deployable anchor structures 20 and 22, respectively. In general, the stent 12 may be constructed to be similar to conventional ureteral stents, as described in the various background patents listed above, the full disclosures of which are incorporated herein by reference.

The evertable, lubricious sleeve will typically be a thin polymer sleeve which can be collapsed and packed into the lumen of the stent 12, typically with a distal end 26 located near or emerging from the distal end 16 of the stent. The proximal end 28 of the sleeve 14 will usually be positioned some distance proximally from the distal end 16 of the stent.

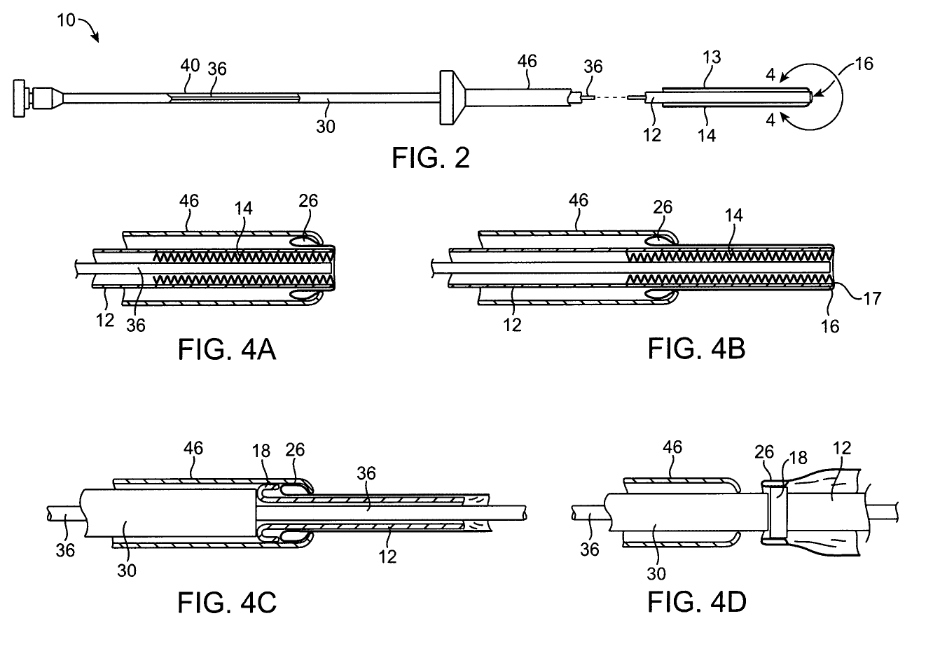

Pusher tube 30 has a distal end 32 adapted to engage and push against the proximal end 18 of the stent 12. A luer or other fitting 34 is found at the proximal end of the pusher tube. A core wire 36 has a length generally equal to the combined lengths of the pusher tube 30 and stent 12 so that the core wire may be passed through a central passage 40 (FIG. 2) of the pusher tube as well as the lumen of stent 12. The core wire serves to maintain the anchor structures 20 and 22 of the stent in a generally linear configuration as the stent is introduced through the body lumen.

An outer positioning tube 46 is provided to help position the remaining components of the stent delivery system 10 for deployment. As will be described in more detail below, the central positioning tube 46, for example, may be placed through the working channel of an endoscope or other cannula or placement device. The fully assembled stent delivery system 10 is illustrated in FIG. 2. The pusher tube 30 is placed within the outer positioning tube 46 with the core wire 36 passing through the central passage 40. Core wire 36 passes into the lumen of stent 12 with the lubricious sleeve 14 being pulled from the distal end 16 of the stent.

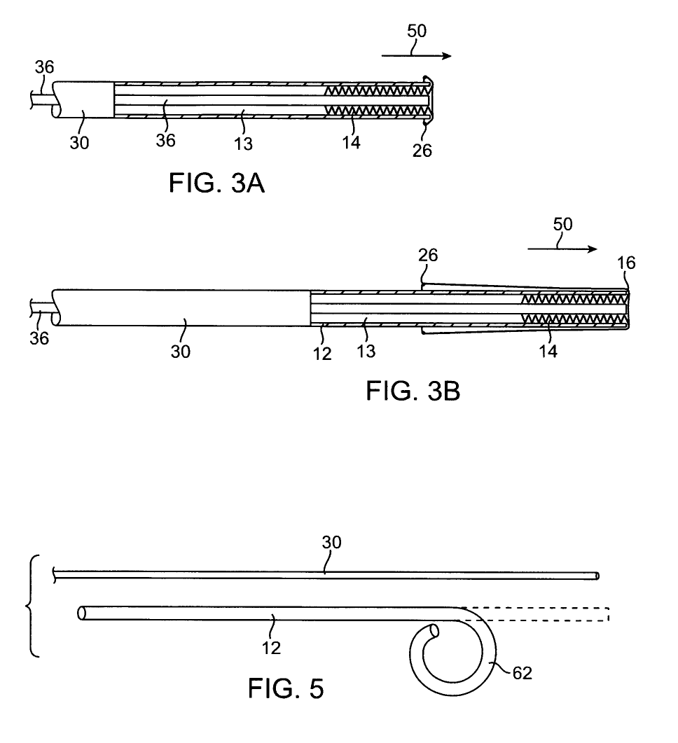

Referring now to FIGS. 3A and 3B, the lubricious sleeve 14 will typically be packed in stent lumen 13, usually about the core wire 36. Distal end 26 of the lubricious sleeve may be held stationary (as described in more detail below) as the stent 12 is advanced distally in the direction of arrow 50. Thus, the packed portion of the sleeve will be drawn forwardly or distally within the stent lumen 13 and will evert or turn rearwardly over the distal tip 16 of the stent, as best shown in FIG. 3B.

The forward or distal end 26 of the lubricious sleeve 14 may be immobilized or held stationary in a variety of ways. For example, it would be possible to provide a very long lubricious sleeve so that it would pass over the entire combined lengths of the stent and pusher tube so that it could be manually held by the physician as the pusher tube is advanced from outside the patient. The distal end 26 of the lubricious sleeve 14, however, is preferably immobilized by releasable attachment to the outer positioning tube 46, as illustrated in FIGS. 4A-4D. The distal end 26 may be formed into an enlarged ring which is received between the exterior of the stent 12 and the interior of the positioning tube 46. As shown in FIG. 4A, almost the entire lubricious sleeve 14 remains packed within the stent 12 about the core wire 36. The stent delivery system 10 would generally be in this configuration when it is introduced through the body lumen prior to any deployment of the stent.

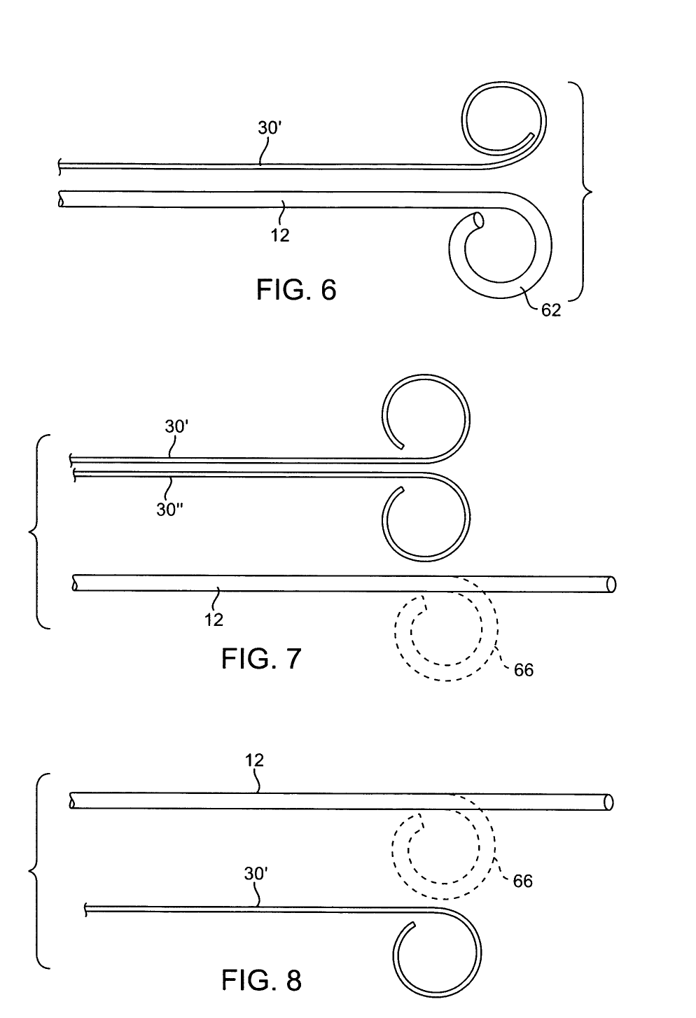

As the stent 12 is advanced distally, as shown in FIG. 4B, the distal end 16 of the stent engages an everting fold line 17 formed in the lubricious sleeve 14. The distal end 26 of the sleeve 14 remains immobilized and captured by the positioning tube 46. As the proximal end 18 of the stent 12 nears the end of the positioning tube 46, as shown in FIG. 4C, a flange or other raised structure on the stent will engage the distal end of the sleeve 26. As the distal end 18 is expelled from the outer positioning tube 46 by the pusher tube 30, as shown in FIG. 4D, the distal end 26 of the lubricious sleeve 14 will also be expelled and freed from the positioning tube. Thus, it will be possible (if desired) to draw on the proximal end of the sleeve 14 to pull the sleeve from over the stent 12 and remove it from the body lumen. As shown in FIGS. 5-8, anchoring structures may be formed in the distal ends of the stent 12 in a variety of ways. As shown in particular in FIG. 5, a stent 12 may have at least one end pre-shaped into a loop or coil 62. A straight core wire 30 may be advanced through a lumen of the stent 12 in order to straighten the stent as shown in broken line. Instead of using a straight core wire 30, a core wire 30′ having a shaped distal end which is opposite to the distal end 62 of the stent 12 may be employed. The core wire 30′ may thus be less stiff than a corresponding straight core wire 30 shown in FIG. 5.

Referring now to FIG. 7, a stent 12 having a straight configuration, i.e. without any curves when unconstrained, may be deformed into a loop or other non-linear configuration. For example, a pair of similar but oppositely coiled core wires 30′ and 30″ may be simultaneously placed in the lumen of the stent 12. While both core wires are present, and arranged to apply forces in opposite directions, the stent 12 will remain straight. By removing either core wire, the stent will be deformed to have a coil end 66, as shown in broken line.

FIG. 8 shows a system employing a coil wire 30′ having a pre-shaped coil at its distal end. By selectively placing the coil wire 30′ into a generally linear stent 12, the stent may be selectively deformed to have a coil end 66 after the stent has been positioned within the kidney or other target body lumen.

It will be appreciated that anchoring structures may be provided on and/or formed within either or both ends of the stents 12 of the present invention in a variety of ways.

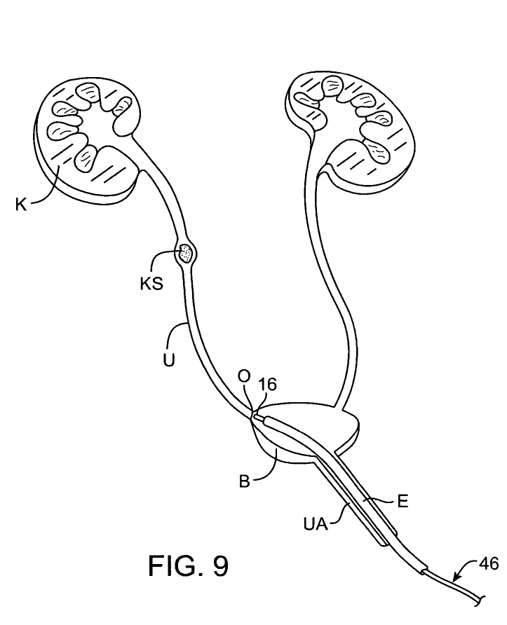

Referring now to FIG. 9, use of the stent deployment system 10 of the present invention for delivering a ureteral stent to a ureter U between a kidney K and bladder B will be illustrated. The outer positioning tube 46 is introduced through a conventional endoscope E through the urethra UA so that the distal end 16 of the stent 12 is located adjacent uretero-vesical orifice O.

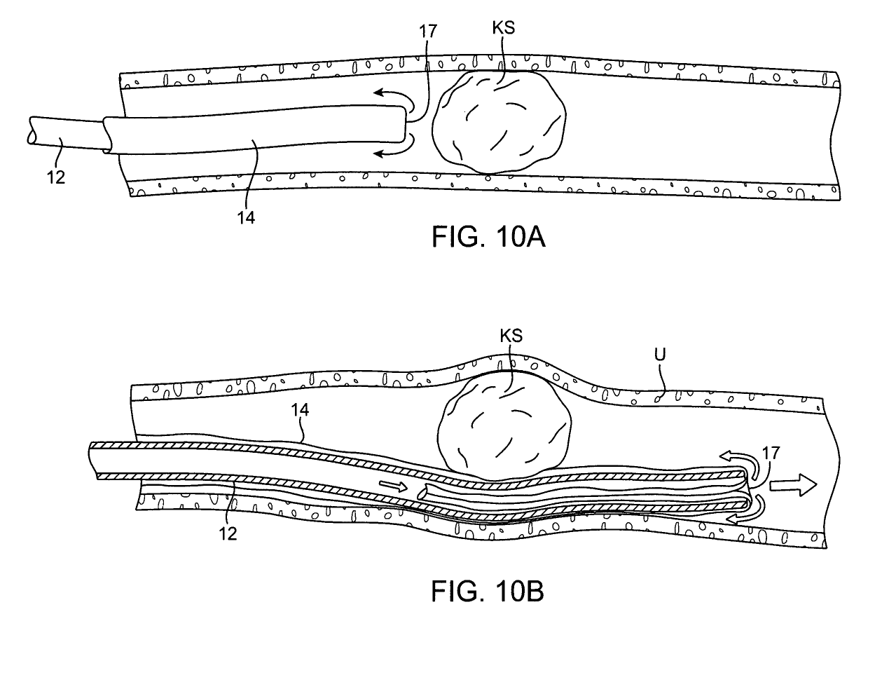

Referring now to FIGS. 10A and 10B, the pusher tube 30 (not illustrated) is advanced against the proximal end of the stent so that the fold line 17 formed by the lubricious sleeve 14 as it everts advances toward the kidney stone KS. As the everting end engages the kidney stone KS, as shown in FIG. 10B, the stent 12 will “track” by the kidney stone while the sleeve 14 is deployed against the wall of the ureter U. The lubricious sleeve 14 acts to protect the ureteral wall from being damaged by the stent as it is advanced through the ureter.

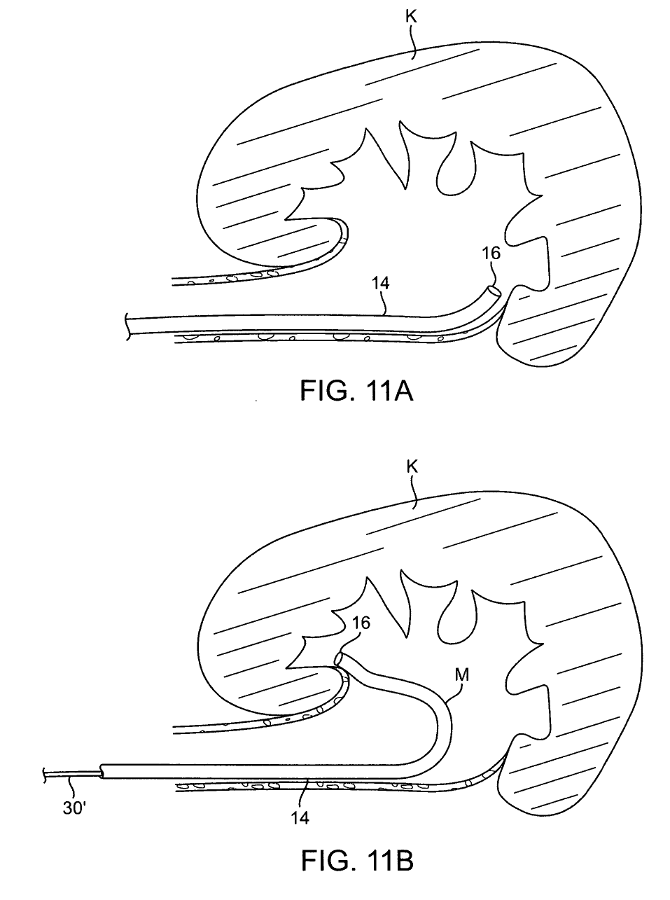

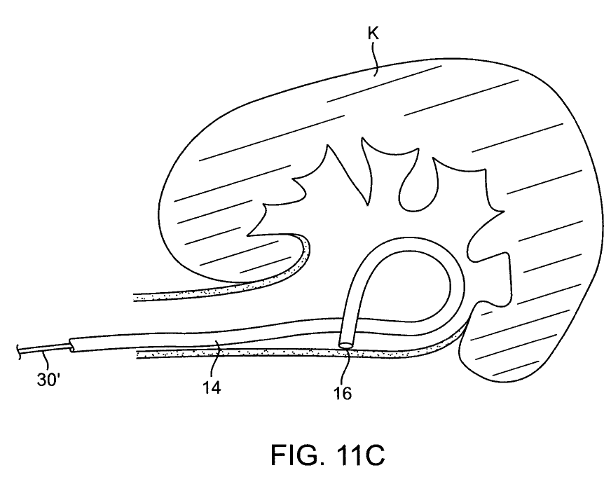

As shown in FIGS. 11A-C, the stent is advanced until the distal end 16, which remains within the lubricious sleeve 14, reaches the inside of the kidney K. Once it is in place, the distal end of the stent can be deformed into an anchoring configuration by any of the techniques described previously. For example, if the stent has a normally linear configuration, a deflection wire 30′ may be exchanged with the core wire to deform the end into an anchoring structure. As shown in FIG. 11B, the deforming wire 30′ has been advanced to a point M causing a J-shaped bend within the stent proximal to the distal end 16. As the wire 30′ is further advanced, the stent completes the coil in the distal end, as shown in FIG. 11C. After the stent has been placed, the lubricious sleeve 14 may be removed by any of the techniques described previously.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for deploying a self-anchoring tubular prosthesis in a body lumen, said method comprising:
advancing the prosthesis in a distal direction through a stationary lubricious sleeve; and
forming at least one anchoring structure at a distal end of the stent after the stent has reached a target location in the body lumen.

2. A method as in claim 1, wherein advancing comprises pushing a pusher member against a proximal end of the prosthesis.

3. A method as in claim 2, further comprising separating the pusher member from the prosthesis and withdrawing the pusher member in a proximal direction after the stent has reached the target location.

4. A method as in claim 1, wherein advancing comprises everting the lubricious sleeve over a distal end of the prosthesis as the stent is advanced, wherein the lubricious sleeve is deployed between the exterior of the prosthesis and the inner wall of the body lumen as the prosthesis is advanced.

5. A method as in claim 4, wherein a distal end of the lubricious sleeve is held stationary relative to the body lumen as the prosthesis is advanced.

6. A method as in claim 5, wherein the distal end of the lubricious sleeve is held by an outer positioning tube which remains stationary within the body lumen.

7. A method as in claim 6, wherein the prosthesis is advanced from a central passage in the outer positioning tube.

8. A method as in claim 4, further comprising pulling a proximal end of the lubricious sleeve from a lumen of the prosthesis to remove the sleeve from the body lumen after the prosthesis has been deployed.

9. A method as in claim 4, further comprising pulling a distal end of the lubricious sleeve from over an exterior of the prosthesis to remove the sleeve from the body lumen after the prosthesis has been deployed.

10. A method as in claim 1, wherein forming the at least one anchoring structure comprises releasing the distal end of the prosthesis from constraint so that the distal end assumes a non-linear configuration.

11. A method as in claim 1, wherein forming the at least one anchoring structure comprises deforming the distal end of the prosthesis to assume a non-linear configuration.

12. A method for deploying a ureteral stent in a ureter, said method comprising:
positioning a stent in an outer positioning tube at a ureteral orifice in a patient's bladder;
pushing on a proximal end of the stent to advance a distal end of the stent into the ureter;
everting an evertable, lubricious sleeve from a lumen of the stent over the distal end of the stent so that the sleeve is laid stationary over the wall of the ureter;
stopping pushing on the proximal end of the stent after a distal portion of the stent has entered a kidney; and
forming at least one anchoring structure in the distal portion to anchor said distal portion within the kidney.

13. A method as in claim 12, wherein everting comprises immobilizing a distal end of the evertable, lubricious sleeve while the stent is advanced against an everting fold of the sleeve.

14. A method as in claim 13, wherein the distal end of the sleeve is immobilized by attachment to the outer positioning tube.

15. A method as in claim 14, further comprising releasing the distal end of the tube from the sleeve and pulling on a proximal end of the sleeve to withdraw the sleeve after the distal end of the stent has entered the kidney.

16. A method as in claim 14, further comprising withdrawing the outer positioning tube to withdraw the sleeve after the distal end of the stent has entered the kidney.

17. A method as in claim 12, wherein forming the at least one anchoring structure comprises releasing the distal end of the stent from constraint so that the distal end assumes a non-linear configuration.

18. A method as in claim 12, wherein forming the at least one anchoring structure comprises deforming the distal end of the stent to assume a non-linear configuration.

19. A stent deployment system comprising:
a tubular stent having a proximal end, a distal end, a lumen therethrough, an exterior surface, and a deployable anchor structure at at least the distal end; and
an evertable, lubricious sleeve adapted to emerge from the distal end of the stent and evert over the exterior surface.

20. A stent deployment system as in claim 19, wherein the sleeve comprises a polymeric tube.

21. A stent deployment system as in claim 20, wherein the polymeric tube has a length in the range from 5 cm to 90 cm, an inner diameter in the range from 2 mm to 12 mm, and a wall thickness in the range from 0.01 mm to 0.05 mm.

22. A stent deployment system as in claim 20, wherein the polymer is a lubricious polymer.

23. A stent deployment system as in claim 20, wherein the polymer is lubricated.

24. A stent deployment system as in claim 20, wherein the polymer is selected from the group consisting of polytetrafluoroethylene (PTFE), polyethylene (PE), perfluoroalkoxy (PFA), polyurethane (PU), perfluoromethylvinylether (MFA), perfluoropropylvinylether (PPVE).

25. A stent deployment system as in claim 24, wherein the polymer comprises tensilized PTFE/PPVE copolymer.

26. A stent deployment system as in claim 19, wherein the tubular stent is not radially expandable.

27. A stent deployment system as in claim 19, wherein the anchor structure comprises a pre-shaped distal end of the stent having a non-linear configuration when unconstrained and a linear configuration when constrained.

28. A stent deployment system as in claim 27, wherein the stent has a proximal anchor structure at its proximal end.

29. A stent deployment system as in claim 28, wherein the proximal anchor structure comprises a pre-shaped distal end of the stent having a non-linear configuration when unconstrained and a linear configuration when constrained.

30. A stent deployment system as in claim 27, further comprising a pusher member.

31. A stent deployment system as in claim 28, wherein the pusher member has a central passage, further comprising a restraining wire receivable through the central passage and the stent lumen to constrain the pre-shaped distal end.

32. A stent deployment system as in claim 32, wherein the restraining wire is a core wire with a length generally equal to the combined lengths of the pusher member and the stent.

33. A stent deployment system as in claim 32, wherein a distal end of the core wire is pre-shaped to offset the shape of the distal end of the stent.

34. A stent deployment system as in claim 31, wherein the wire is a guidewire with a length significantly longer than the combined lengths of the pusher member and the stent.

35. A stent deployment system as in claim 27, further comprising a restraining tube which is received over at least the stent to constrain and straighten the pre-shaped distal end.

36. A stent deployment system as in claim 19, further comprising an outer positioning tube having a central passage which receives the tubular stent.

37. A stent deployment system as in claim 36, wherein a distal end of the evertable, lubricious sleeve is releasably attached to a distal end of the outer positioning tube, whereby the stent can be advanced distally through the outer positioning tube while the sleeve is everted from the lumen of the stent to track over the exterior of the stent.
